SEQUENCE LISTING (2 sequences)

SEQ ID NO: 1 - protein, 1304 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGNVTMNLTTKVANDTKAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGADKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVDWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSERVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERD

SEQ ID NO: 2 - protein, 887 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKT

(19) United States
(12) Patent Application Publication
(10) Pub. No.: US 2025/0000113 A1
(43) Pub. Date: Jan. 2, 2025

(54) METHOD FOR PRODUCING A DAIRY PRODUCT

(71) Applicant: KERRY GROUP SERVICES INTERNATIONAL LTD, Co. Kerry (IE)

(72) Inventor: Jeppe Wegener Tams, Bagsvaerd (DK)

(21) Appl. No.: 18/708,997

(22) PCT Filed: Nov. 10, 2022
(86) PCT No.: PCT/EP2022/081496

§ 371 Date: May 9, 2024

(30) Foreign Application Priority Data
Nov. 12, 2021 (EP) 21207944.6

(51) Int. Cl. A23C 9/12; A23C 3/02; C12N 9/38

(52) U.S. Cl. CPC A23C 9/1206; A23C 3/02; C12N 9/2471; C12Y 302/01023; A23C 2220/10

(57) ABSTRACT

The present invention relates to a method of producing a dairy product comprising galacto-oligosaccharides (GOS) where a milk-based substrate is incubated with a Bifidobacterium de-rived beta-galactosidase having transgalactosylating activity and the beta-galactosidase is heat inactivated.

### REFERENCE TO SEQUENCE LISTING

This application contains a Sequence Listing in computer readable form. The computer readable form is incorporated herein by reference.

### FIELD OF THE INVENTION

The present invention relates to a method of producing a dairy product comprising galacto-oligosaccharides (GOS) where a milk-based substrate is incubated with a beta-galactosidase having transgalactosylating activity and the beta-galactosidase is heat inactivated.

### BACKGROUND OF THE INVENTION

Galactooligosaccharides (GOS) can be produced from lactose through the enzymatic transgalactosylation reaction of a beta-galactosidase. Beta-galactosidase (EC 3.2.1.23) usually hydrolyses lactose to the monosaccharides glucose and galactose. In such hydrolysis reaction, the beta-galactosidase enzyme hydrolyses lactose and transiently binds the galactose monosaccharide in a galactose-enzyme complex. Subsequently, water is used to hydrolyse the covalent galactose-enzyme intermediate resulting in the liberation of galactose and glucose. However, sometimes, in particular at high lactose concentrations, the enzyme instead transfers the galactose to the hydroxyl group of galactose or glucose in a so-called transgalactosylation reaction thus producing GOS.

It has been shown that GOS can promote the growth of bifidobacteria, which are healthy microbes, in the large intestine of humans. Therefore, it is beneficial and desirable to produce milk products comprising high amounts of GOS.

GOS can be produced ex situ from solutions of lactose and added to dairy products as an ingredient. This will add calories and sugars (lactose plus glucose and galactose) to the dairy product, and further the addition of GOS is to be labelled on the final product. This goes against the general “clean label” trend, i.e. the preference of many consumers of “natural” products comprising only ingredients which are perceived as natural with no or few additives.

It is therefore sometimes preferred to produce the GOS in situ, i.e. to make the GOS in the milk product from the lactose already present in the milk-based substrate, e.g. in milk. Which besides generation of the pre-biotic GOS also reduce the sugar content in the finished dairy product since some of the sugar is converted to GOS fiber, e.g. if 20% of the lactose is converted to GOS fiber, this results in 20% reduction of sugar on the label plus minor reduction in amount of calories as GOS fiber has 2 kcal/g whereas sugar has 4 kcal/g. Thus, in situ GOS fiber enriched products fit with current consumer trend to reduce sugar and calories in food products.

In Jørgensen et al. (2001), Appl. Microbiol. Biotechnol. 57: 647-652, WO2001/90317 and WO2013182686, Bifidobacterium bifidum derived beta-galactosidases have been disclosed which are good transgalactosylating enzymes. In Jørgensen et al. (2001), GOS production in 0.4 M lactose solution was performed and the Bifidobacterium bifidum derived beta-galactosidase was inactivated by 5 minutes incubation at 95° C. In WO2013182686, Bifidobacterium bifidum derived beta-galactosidase was used to produce GOS in milk and yoghurt and before quantification of the GOS by HPLC the enzyme was inactivated by heating the samples to 95° C. for 10 minutes.

WO2012/010597 discloses a method of producing GOS wherein the galactosyl acceptor is different from the galactosyl donor. In the examples, a lactose solution is incubated with a Bifidobacterium bifidum derived beta-galactosidase to characterize the enzyme and inactivation is performed by heating to 85° C. for 10 minutes.

In WO2015/086746, the in situ conversion of low-concentration lactose to GOS in a milk-based substrate such as milk was studied using a Bifidobacterium bifidum derived beta-galactosidase. It was found that the content of GOS produced in such in situ milk-based dairy application is not stable over time and is highly dependent on no or very low amounts of residual beta-galactosidase activity in the milk-based product under normal storage conditions. It was found that the Bifidobacterium bifidum derived beta-galactosidase tested was highly stabilized in milk compared to a buffered solution and that a surprisingly high combination of treatment time and temperature was required to completely inactivate the Bifidobacterium bifidum derived beta-galactosidase in milk. It was found that, e.g., pasteurization at 95° C. for 6 minutes did not sufficiently inactivate the enzyme. It was found that heat treatment at a temperature of above 130° C. may create of-flavours, denaturation and browning of the product, whereas heat treatment at a temperature below 90° C. results in a holding time which may not be compatible with an industrial process. Specific inactivation conditions were defined which were needed to completely inactivate the Bifidobacterium bifidum derived beta-galactosidase and ensure stable GOS over time, namely heat-treatment of the GOS-comprising milk-based substrate at a temperature T in the range of 90-130° C. for a period of time of at least x seconds, where x=153,377,215,802.265 e^{-0.20378144T}. This corresponds to, e.g., treatment at 90° C. for 28 minutes, treatment at 95° C. for 10 minutes or treatment at 130° C. for 48 seconds.

WO2015/132402 discloses a method of producing a GOS-containing milk product by contacting the milk-derived composition with an enzyme having transgalactosylating activity, which may be derived from Bifidobacterium bifidum, at a temperature of at most 10° C. and inactivating and/or removing at least some of the enzyme. The inactivation is to preferably provide a reduction of at least 90% of the beta-galactosidase activity and heating to at least 90° C. for at least 10 minutes, perhaps involving low temperature e.g. starting from 80° C., is suggested. In the examples, heat inactivation of a Bifidobacterium bifidum derived enzyme having transgalactosylating activity is at 95° C. for 10 minutes.

In WO2018/210821, a Bifidobacterium bifidum derived lactase was used for production of GOS in milk or in 60% skim milk powder and the enzyme was inactivated by adding acetic acid and heating to 90° C. for 5 minutes. Fully or partly inactivating by pasteurization, ESL treatment, UHT treatment or spray-drying is also suggested.

In WO2020/117548, a low lactose milk-based product having GOS fiber is produced by treating a milk-based substrate with a transgalactosylating enzyme to provide GOS, deactivating the transgalactosylating enzyme, adding a lactase to degrade the remaining lactose and deactivating the lactase. A Bifidobacterium bifidum lactase was used for the transgalactosylation step and the enzyme was inactivated by pasteurization at 95° C. for 5 minutes or UHT pasteurization including heating to 142° C. for 1-2 seconds. Deactivation by heat treatment at 70 to 95° C., preferably at about 95° C., for 5 to 30 minutes or at 135 to 150° C. for 2 to 15 seconds is also suggested.

Hsu et al., 2007, J. Agric. Food Chem. 55:2225-2230, studied GOS formation with a beta-galactosidase from Bifidobacterium longum in a 40% lactose solution. The enzyme was inactivated by heating at 100° C. for 10 minutes.

### SUMMARY OF THE INVENTION

The present inventor has surprisingly found that commercial transgalactosylating Bifidobacterium derived beta-galactosidase products can be inactivated at lower temperatures than the temperatures used in the prior art. Even if not completely inactivated at those temperatures, the inactivation achieved is good enough to ensure stable GOS levels in commercially relevant applications. Lower inactivation temperatures are generally preferred in the industry to avoid off-flavors of the final dairy products.

The present inventor has surprisingly found that inactivation of a Bifidobacterium derived beta-galactosidases at 90° C. and 80° C. results in minimum residual activity when inactivating for approximately 5 minutes and 8 minutes, respectively, and then higher residual activity is seen when inactivating for a longer time at these temperatures. Whereas inactivation at 70° C. and 75° C. results in a continuous decrease in residual activity when increasing the inactivation time from 5 minutes to 15 minutes. Inactivation at 75° C. results in a very low residual activity when using an inactivation time of at least 12 minutes of a GOS-enriched milk comprising active enzyme.

The present invention therefore in one aspect provides a method for producing a dairy product comprising galacto-oligosaccharides (GOS), said method comprising:
a. incubating a milk-based substrate with a Bifidobacterium derived beta-galactosidase having transgalactosylating activity to produce GOS, and
b. heat-treating the milk-based substrate comprising beta-galactosidase and GOS at a temperature of 72-78° C. for at least 12 minutes so that the residual activity of the beta-galactosidase is at most 0.1%.

Such inactivation scheme is useful following in situ GOS production at low temperature for, e.g., 12-30 hours.

Surprisingly, the GOS production may also be performed at quite high temperature such as 55-60° C. for short time such as for 30 minutes to 4 hours. Shortening of the incubation time is sometimes desired, e.g., to save tank capacity.

The present inventor has also surprisingly found that a “one step GOS production and inactivation” scheme can be applied which provides sufficient inactivation of the enzyme in applications where the dairy product is not to be stored for prolonged time at ambient temperature or where a later process step is included which will further inactivate the enzyme, e.g., a UHT treatment or a drop in pH such as in yoghurt production.

The present invention therefore in another aspect provides a method for producing a dairy product comprising galacto-oligosaccharides (GOS), said method comprising:
a. adding to a milk-based substrate a Bifidobacterium derived beta-galactosidase having transgalactosylating activity, and
b. incubating at a temperature of 62-66° C., preferable 62.5-65.5° C., more preferably 63-65° C., for at least 2 hours, preferably at least 3 hours, more preferably at least 4 hours, to produce GOS and at the same time inactivate the beta-galactosidase so that after the incubation, the residual activity of the beta-galactosidase is at most 1%, preferably at most 0.5%.

### DETAILED DESCRIPTION OF THE INVENTION

Dairy products comprising galacto-oligosaccharides (GOS) are highly desirable. GOS can be disaccharides (DP2) (except lactose which is per definition not GOS), trisaccharides (DP3), tetrasaccharides (DP4), pentasaccharides (DP5) or longer oligosaccharides. DP is short for Degree or Polymerization.

In the context of the present invention, the term “DPx+GOS” means the sum of GOS molecules having DPx or higher DP. For example, the term “DP3+” means the sum of GOS molecules having DP3, DP4, DP5, DP6, etc.

DP3+GOS are thus GOS having a degree of polymerization of at least 3. I.e., DP3+GOS are trisaccharides, tetrasaccharides, pentasaccharides and longer oligosaccharides. DP3+GOS are per definition fiber and can be claimed as such. DP3+GOS are therefore also referred to as GOS fiber.

The dairy industry has an interest in claiming contents of GOS in their dairy products as well as contents of fiber.

High contents of in situ produced GOS or in situ produced GOS fiber (DP3+GOS) are of particular interest.

The amount of GOS may be determined in wt % (weight %, also sometimes denoted as % (w/w)) out of total carbohydrates.

The amount of GOS may be determined using the method of the Examples of the present application.

The total carbohydrates in this context are the total free carbohydrates, i.e. the carbohydrates which are not covalently bound to proteins.

In milk products where the carbohydrates have not been manipulated, e.g., by enzymatic treatment, the vast majority of free carbohydrates is lactose.

Bifidobacterium derived beta-galactosidases, like other multi-domain beta-galactosidases, can be modified by C-terminal truncation to obtain a higher ratio of transgalactosylating to hydrolysing activity (see, e.g., Jorgensen et al. (2001), Appl. Microbiol. Biotechnol. 57: 647-652; WO2001/90317; WO2013182686). Alternatively, a higher ratio of transgalactosylating to hydrolysing activity of such beta-galactosidases can be obtained by incubation with a reducing sugar, e.g., glucose, resulting in glycation of some amino acid residues (WO 2018/210820; WO 2018/210821).

Complete inactivation of Bifidobacterium derived beta-galactosidases in milk-based substrates such as milk has been shown to require a combination of high temperature and incubation time, e.g., 90° C. for 28 minutes, 95° C. for 10 minutes or 130° C. for 48 seconds (WO2015/086746).

The present study confirms that incubation at, e.g., 90° C. for 15 minutes does not sufficiently inactivate Bifidobacterium derived beta-galactosidases in milk-based substrates. However, surprisingly, incubation at 75° C. for at least 12 minutes results in a very low or not detectable residual activity. Lower inactivation temperatures are desirable as higher temperatures introduce off-flavors and color formation in the dairy product.

Very low or no residual beta-galactosidase activity is particularly important to maintain a stable level of in situ produced GOS in a dairy product.

The present invention provides commercially relevant methods for in situ GOS production using a Bifidobacterium derived beta-galactosidase having transgalactosylating activity where the beta-galactosidase is inactivated at relatively low temperature.

### Inactivation at 72-78° C.

Incubation at 75° C. for at least 12 minutes is shown herein to result in the residual activity being sufficiently low to ensure no or minimal GOS degradation during prolonged storage of the dairy product in particular at low temperature.

The present invention therefore in one aspect provides a method for producing a dairy product comprising galacto-oligosaccharides (GOS), said method comprising:
a. incubating a milk-based substrate with a Bifidobacterium derived beta-galactosidase having transgalactosylating activity to produce GOS, and
b. heat-treating the milk-based substrate comprising beta-galactosidase and GOS at a temperature of 72-78° C. for at least 12 minutes so that the residual activity of the beta-galactosidase is at most 0.1%.

In step a of said method, the incubation may be performed at 0-15° C., preferably at 4-10° C. Incubation at low temperature is preferred in some applications to not reduce the quality of the milk-based substrate such as the milk. The incubation in step a may be performed for 12-30 hours, preferably for 16-25 hours.

In other applications, a short incubation at quite high temperature is preferred. Therefore, the incubation in step a may alternatively be performed at 50-60° C., preferably at 56-60° C. The incubation may be performed for 30 minutes to 4 hours, preferably for 45 minutes to 2 hours.

After step a, the milk-based substrate preferably comprises at least 10 wt % DP2+GOS, more preferably at least 20 wt %, even more preferably at least 30 wt % DP2+GOS, out of total carbohydrates. More preferably, the milk-based substrate comprises 10-70 wt % DP2+GOS, preferably 20-60 wt %, more preferably 30-50 wt % DP2+GOS, out of total carbohydrates

After step a, the milk-based substrate preferably comprises at least 10 wt % DP2 GOS, more preferably at least 20 wt %, even more preferably at least 30 wt % DP2 GOS, out of total carbohydrates. More preferably, the milk-based substrate comprises 10-70 wt % DP2 GOS, preferably 20-60 wt %, more preferably 25-40 wt % DP2 GOS, out of total carbohydrates.

After step a, the milk-based substrate preferably comprises at least 1 wt % DP3+GOS, more preferably at least 2 or at least 3 wt %, even more preferably at least 4 or at least 5 wt % DP3+GOS, out of total carbohydrates. More preferably, the milk-based substrate comprises 1-50 wt % DP3+GOS, preferably 2-40 wt %, more preferably 5-30 wt % DP3+GOS, out of total carbohydrates.

The amount of the DP3 GOS β-D-Galp-(1→3)-β-D-Galp-(1→4)-D-Glcp is measured in the Examples of the present application. It is a trisaccharide GOS where a galactose is linked to lactose via a beta 1-3 bond in the non-reducing end of lactose and it is also referred to as Gal_b1-3_Lac.

After step a, the milk-based substrate preferably comprises at least 1 wt % β-D-Galp-(1→3)-β-D-Galp-(1→4)-D-Glcp GOS, more preferably at least 2 or at least 3 wt %, even more preferably at least 4 or at least 5 wt % β-D-Galp-(1→3)-β-D-Galp-(1→4)-D-Glcp GOS, out of total carbohydrates. More preferably, the milk-based substrate comprises 1-30 wt % β-D-Galp-(1→3)-β-D-Galp-(1→4)-D-Glcp GOS, preferably 2-20 wt %, more preferably 3-15 wt % β-D-Galp-(1→3)-β-D-Galp-(1→4)-D-Glcp GOS, out of total carbohydrates.

In step b of the method, the heat treatment may be performed at a temperature of 73-78° C., preferably 73-77° C., more preferably 74-77° C., even more preferably 74-76° C., and most preferably 74.5-75.5° C. The heat treatment is performed to inactivate the beta-galactosidase. The heat treatment may be performed for at least 13 minutes, preferably at least 14 minutes, more preferably at least 15 minutes, even more preferably at least 30 minutes, most preferably at least 45 minutes.

The heat treatment of step b of the method may be performed for 12 minutes to 6 hours, such as for 13 minutes, 14 minutes, 15 minutes, 30 minutes or 45 minutes to 6 hours, preferably for 12 minutes to 4 hours, such as for 13 minutes, 14 minutes, 15 minutes, 30 minutes or 45 minutes to 4 hours, more preferably for 12 minutes to 2 hours, such as for 13 minutes, 14 minutes, 15 minutes, 30 minutes or 45 minutes to 2 hours, even more preferably for 12 minutes to 1 hour, such as for 13 minutes, 14 minutes, 15 minutes, 30 minutes or 45 minutes to 1 hour, and most preferably for 30 minutes to 1 hour.

In an industrial setting, the enzymatic treatment of the milk-based substrate (step a) may be performed batch-wise in a tank. And the heat treatment to inactivate the enzyme (step b) may be performed in another tank. The time it takes to fill and empty such tank may be 30-60 minutes. In that case, it is for practical reasons not possible to perform the heat treatment for e.g. 15 minutes.

In some embodiments of the invention, the heat treatment in step b is performed for the time it takes to fill and empty the tank in which the heat treatment takes place.

After step b, the residual activity of the Bifidobacterium derived beta-galactosidase is at most 0.1%, preferably at most 0.05%, more preferably at most 0.01%. The residual activity is calculated as the activity after step b in percent of the activity before step a. Preferably, the residual activity is calculated as the activity immediately after step b in percent of the activity before step a. The skilled person will know how to determine the residual activity. It may be determined using the methods of the Examples of the present application.

It is important that the residual activity is low enough to ensure that during storage, such as during storage at room temperature, the GOS are not degraded or only minimally degraded.

After step b of the method, the amount of β-D-Galp-(1→3)-β-D-Galp-(1→4)-D-Glcp GOS is preferably reduced by at most 15%, more preferably at most 10%, after 3 months storage of the dairy product at 20° C.

### One-Step GOS Production and Inactivation

Further, a “one step GOS production and inactivation” at 62-66° C. for at least 2 hours is shown herein to provide sufficient inactivation of the enzyme to ensure a sufficiently stable GOS level in some applications where the dairy product is not to be stored for prolonged time or where a later process step is included which will further inactivate the enzyme, e.g., a UHT treatment or a drop in pH such as in yoghurt production.

The present invention therefore in another aspect provides a method for producing a dairy product comprising galacto-oligosaccharides (GOS), said method comprising:
a. adding to a milk-based substrate a Bifidobacterium derived beta-galactosidase having transgalactosylating activity, and
b. incubating at a temperature of 62-66° C., preferable 62.5-65.5° C., more preferably 63-65° C., for at least 2 hours, preferably at least 3 hours, more preferably at least 4 hours, to produce GOS and at the same time inactivate the beta-galactosidase so that after the incubation, the residual activity of the beta-galactosidase is at most 1%, preferably at most 0.5%.

The residual activity is the activity after step b in percent of the activity before step a.

Preferably, after step b, the milk-based substrate comprises at least 8 wt % DP2+GOS, preferably 8-70 wt %, more preferably 8-50 wt % DP2+GOS, out of total carbohydrates.

Preferably, after step b, the milk-based substrate comprises at least 2 wt % DP2 GOS, preferably 2-60 wt %, more preferably 2-40 wt % DP2 GOS, out of total carbohydrates.

Preferably, after step b, the milk-based substrate comprises at least 5 wt % DP3+GOS, preferably 5-50 wt %, more preferably 5-30 wt % DP3+GOS, out of total carbohydrates.

Preferably, after step b, the milk-based substrate comprises at least 1 wt % β-D-Galp-(1→3)-β-D-Galp-(1→4)-D-Glcp GOS, preferably 1-30 wt %, more preferably 3-15 wt % R-D-Galp-(1→3)β-D-Galp-(1→4)-D-Glcp GOS, out of total carbohydrates.

Step b is preferably performed for 2-6 hours, more preferably 3-5 hours.

### General Embodiments for all Aspects of the Invention

Preferably, the dairy product is milk, e.g., skim milk, low fat milk, whole milk, UHT milk, ESL milk or HTST milk, or chocolate milk or yoghurt or whey.

In some embodiments of the methods of the invention, the dairy product is obtained from the milk-based substrate without the addition of anything except for beta-galactosidase.

In step a of the methods of the invention, GOS are produced in situ from the lactose present in the milk-based substrate.

The milk-based substrate comprises lactose, preferably 3-10 wt % lactose, more preferably 4-5.5 wt % lactose.

In some embodiments, the milk-based substrate which is incubated with beta-galactosidase in step a does not comprise added lactose. In other embodiments, the milk-based substrate which is incubated with beta-galactosidase in step a comprises added lactose.

Preferably, the milk-based substrate is milk, e.g., raw milk, skim milk, low fat milk or whole milk, reconstituted milk powder, e.g., reconstituted skim milk powder, or chocolate milk or whey. The milk-based substrate may be a combination of, e.g., milk and milk powder, milk and whey or reconstituted milk powder and whey.

In some embodiments, the milk-based substrate is obtained from raw milk and has not been largely modified in composition by addition and/or withdrawal of constituents except for adjustment of fat content and/or water.

The milk-based substrate may have been pasteurized before step a.

In some embodiments, the milk-based substrate is obtained from raw milk and has not been modified in composition except for adjustment of fat content and/or water.

Adjustment of fat content and/or water may have been done by any method known in the art, e.g., by centrifugation, evaporation, condensation, ultrafiltration, nanofiltration, freeze-drying, spray-drying, reconstitution by addition of water, etc.

In some embodiments, e.g., the protein content or the lactose content may also have been slightly adjusted by, e.g., the dairy company, e.g. to obtain a dairy product having a well-defined “standard” composition.

In some embodiments, the whey protein to casein ratio in the milk-based substrate has not been altered. In some embodiments, the whey protein to casein ratio in the milk-based substrate is the same as in milk.

In some embodiments, no milk protein has been added to or removed from the milk-based substrate. In other embodiments, the milk protein content in the milk-based substrate has only been slightly adjusted, e.g. by addition/removal of at most 20%, preferably at most 10%, of the milk protein originally present. This means that the overall milk protein content is largely the same as in raw milk, except for relative changes caused by removal or addition of fat or water.

In some embodiments, at most 20%, preferably at most 10%, milk protein has been added to or removed from the milk-based substrate compared to the milk protein originally present.

In some embodiments, the milk-based substrate comprises a total amount of at least 10 wt % milk protein, such as at least 15 wt % or at least 20 wt %, milk protein.

After step b of the methods of the invention, the dairy product may be stored. It may be stored, e.g., at room temperature, or it may be stored, e.g., at below 10° C., such as at 0-10° C., which will prolong the storage stability of the dairy product and give it a longer shelf-life.

After step b of the methods of the invention, the dairy product may be subjected to treatments known in the art, such as, e.g., ESL treatment, HTST treatment or UHT treatment. Such treatment may be performed to reduce the microbial count in the dairy product to ensure a long shelf-life. The microbial count may not be sufficiently reduced by the inactivation according to the methods of the invention. On the other hand, an ESL treatment, an HTST treatment or a UHT treatment may not sufficiently inactivate the Bifidobacterium derived beta-galactosidase to ensure minimal degradation of the GOS produced. However, an ESL treatment, an HTST treatment or a UHT treatment may contribute to further inactivate the enzyme when using a method of the invention.

After step b of the methods of the invention, a drying, preferably a spray-drying, of the dairy product may be performed to obtain a dried dairy product such as a milk powder.

The dairy product produced by a method of the invention can be consumed as is. Or it can be used as an ingredient in production of, e.g., a protein bar, a milk drink, an infant formula or a yogurt comprising in situ produced GOS.

After step b of the methods of the invention, the dairy product may be fermented with a bacterial culture to provide a fermented dairy product comprising in situ produced GOS. The fermentation with the bacterial culture is preferably performed at a temperature of 37-45° C.

The fermentation with a bacterial culture will result in a drop in pH which will not in itself sufficiently inactivate the Bifidobacterium derived beta-galactosidase to ensure minimal degradation of the GOS produced. However, such drop in pH will contribute to further inactivate the enzyme when using a method of the invention.

### Beta-galactosidase Having Transgalactosylating Activity

In the methods of the invention, the milk-based substrate is contacted with a beta-galactosidase having transgalactosylating activity.

Beta-galactosidases from glycoside hydrolase family 2 (GH2) are exo-acting enzymes, which hydrolyse terminal non-reducing beta-D-galactose residues in beta-D-galactosides, e.g. lactose is hydrolysed to galactose and glucose. They belong to the enzyme class EC 3.2.1.23 with the official name beta-D-galactoside galactohydrolase. A common name used for this enzyme is lactase, as lactose is the common industrial substrate. Besides hydrolysing, this enzyme class is also able to transfer galactose to other sugars and thereby make galacto-oligosaccharides (GOS). The different GH2 enzymes have various preferences for hydrolytic or beta-galactosidase activity and transgalactosylase activity and the preference can be expressed in terms of their GOS production ability, such as by the ratio of transgalactosylating to hydrolysing activity.

In the present context, the term “beta-galactosidase” means any glycoside hydrolase having the ability to hydrolyse the disaccharide lactose into its constituent galactose and glucose monomers. Enzymes assigned to subclass EC 3.2.1.108, also called lactases, are also considered a beta-galactosidase in the context of the present invention. In the context of the invention, the lactose hydrolysing activity of the beta-galactosidase may be referred to as its lactase activity or its beta-galactosidase activity.

In the context of the present invention, the Bifidobacterium derived beta-galactosidase having transgalactosylating activity preferably belongs to the enzyme class EC 3.2.1.23 or EC 3.2.1.108, more preferably 3.2.1.23. The Bifidobacterium derived beta-galactosidase having transgalactosylating activity preferably belongs to glycoside hydrolase family 2 (GH2), more preferably to the glycoside hydrolase family GH2_5.

In certain applications, combinations of polypeptides having predominantly transgalactosylating activity and predominantly hydrolysing activity may be contemplated. This may be especially useful when there is a desire to reduce residual lactose after treatment with the beta-galactosidase having transgalactosylating activity, for example at low lactose levels.

When considering the reaction of the enzyme in e.g. milk, carbohydrates are initially present in the form of lactose, a disaccharide composed of galactose and glucose that is found in milk. In the formation of GOS, successive galactose molecules are added to lactose, and then after prolonged incubation a mixture of the various carbohydrates is present (glucose, galactose and ˜30 different di- and polysaccharides).

The term “disaccharide” as used herein means two monosaccharide units bound together by a covalent bond known as a glycosidic linkage formed via a dehydration reaction, resulting in the loss of a hydrogen atom from one monosaccharide and a hydroxyl group from the other.

As used herein, the terms “beta-galactosidase having transgalactosylating activity”, “transgalactosylating beta-galactosidase”, “enzyme having transgalactosylating activity”, “transgalactosylating enzyme”, “polypeptide having transgalactosylating activity”, “transgalactosylating polypeptide” and “transgalactosylase” are used interchangeably to mean an enzyme that is able to transfer galactose to the hydroxyl groups of D-galactose (Gal) or D-glucose (Glc) whereby galactooligosaccharides are produced. In one embodiment, transgalactosylase activity is identified by reaction of the enzyme on lactose in which the amount of galactose generated is less than the amount of glucose generated at a given time.

More particularly, the transgalactosylase activity (or preference for an enzyme to either hydrolyze lactose or to produce GOS) can be evaluated as the amount of glucose minus galactose generated at any given time during reaction or by direct quantification of GOS generated during the reaction. This measurement may be performed by one of several ways including the methods shown in the Examples herein.

When evaluating the transgalactosylating activity versus hydrolysing activity of an enzyme, the hydrolysing activity may be measured as the concentration of galactose ([Gal]) generated at any time point during the reaction.

The transgalactosylating activity, i.e. the transfer of a galactose moiety to a molecule other than water, may be measured as ([Glc]˜[Gal]) generated at any given time.

Thus, the ratio of transgalactosylating to hydrolysing activity in the context of the present invention is determined as ([Glc]˜[Gal])/[Gal].

Preferably, a Bifidobacterium derived beta-galactosidase to be used in a method of the invention has a ratio of transgalactosylating to hydrolysing activity of at least 1, preferably at least 2, at least 3 or at least 4, when measured in a 60% solution of skim milk powder after 24 hours at 5° C. using an enzyme concentration of 350 LAU(C) per g lactose.

The ratio of transgalactosylating to hydrolysing activity is calculated as ([Glc]˜[Gal])/[Gal], where [Glc] is the relative concentration of glucose and [Gal] is the relative concentration of galalctose after the skim milk powder solution and the beta-galactosidase has been incubated for 24 hours at 5° C. The concentration of glucose and galactose, respectively, is preferably measured using HPLC.

Preferably, a Bifidobacterium derived beta-galactosidase to be used in a method of the invention has a ratio of transgalactosylation activity of at least 150%. The ratio of transgalactosylation activity is determined according to Method 3 of WO2013182686, which is incorporated herein by reference.

Any Bifidobacterium derived beta-galactosidase having transgalactosylating activity can be used in a method of the invention. In a preferred embodiment, the Bifidobacterium derived beta-galactosidase is derived from B. animalis, B. bifidum, B. breve, B. infantis, B. lactis, or B. longum. In a more preferred embodiment, the Bifidobacterium derived beta-galactosidase is derived from Bifidobacterium bifidum.

In a preferred embodiment, the Bifidobacterium derived beta-galactosidase has been C-terminally truncated compared to the wild-type enzyme from which is it derived.

In a preferred embodiment, the Bifidobacterium derived beta-galactosidase has an amino acid sequence which is at least 50%, such as at least 60%, at least 70%, at least 80%, at least 90%, at least 95%, at least 98%, at least 99%, at least 99.5% or 100% identical to any of SEQ ID NOs: 1 or 2.

For purposes of the present invention, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the -nobrief option must be specified in the command line. The output of Needle labeled “longest identity” is calculated as follows:

(Identical Residues×100)/(Length of Alignment−Total Number of Gaps in Alignment)

In a preferred embodiment, the Bifidobacterium derived beta-galactosidase has an amino acid sequence which is at least 50%, such as at least 60%, at least 70%, at least 80%, at least 90%, at least 95%, at least 98%, at least 99%, at least 99.5% or 100% identical to SEQ ID NO: 1.

In another preferred embodiment, the Bifidobacterium derived beta-galactosidase has an amino acid sequence which is at least 50%, such as at least 60%, at least 70%, at least 80%, at least 90%, at least 95%, at least 98%, at least 99%, at least 99.5% or 100% identical to SEQ ID NO: 2.

The Bifidobacterium derived beta-galactosidase preferably has a length of 850-1500 amino acids, preferably 850-1350 amino acids, more preferably 880-1350, 880-1320 or 885-1310 amino acids.

In preferred embodiment, the Bifidobacterium derived beta-galactosidase has an amino acid sequence which is at least 50%, such as at least 60%, at least 70%, at least 80%, at least 90%, at least 95%, at least 98%, at least 99%, at least 99.5% or 100% identical to SEQ ID NO: 1 and a length of 850-1500 amino acids, preferably 1250-1500 amino acids, more preferably 1250-1350 or 1290-1350 amino acids, even more preferably 1300-1305 amino acids such as 1302 or 1304 amino acids.

In another preferred embodiment, the Bifidobacterium derived beta-galactosidase has an amino acid sequence which is at least 50%, such as at least 60%, at least 70%, at least 80%, at least 90%, at least 95%, at least 98%, at least 99%, at least 99.5% or 100% identical to SEQ ID NO: 2 and a length of 850-1500 amino acids, preferably 850-1250 amino acids, more preferably 880-1215 amino acids, even more preferably 885-888 amino acids.

The Bifidobacterium derived beta-galactosidase may have been modified by glycation of at least one lysine and/or arginine residue.

“Glycation” as used herein refers to the covalent attachment of a carbohydrate to a protein. Carbohydrate attachment may be via a side chain of, e.g., arginine, lysine, or N-terminal of the enzyme. Preferably, the carbohydrate attachment is via a side chain of arginine or lysine.

The Bifidobacterium derived beta-galactosidase may have been modified by glycation by being contacted with a reducing sugar, preferably fructose, glucose, galactose, or lactose, for a time and temperature sufficient to produce a polypeptide modified by glycation. Preferably, it has been modified by glycation by being contacted with 30-90 wt %, preferably 40-70 or 40-55 wt %, of a reducing sugar, preferably fructose, glucose, galactose or lactose, more preferably fructose, glucose or galactose, even more preferably glucose, at pH 4-7 for a time of 3-100 hours, preferably 40-100, 50-90 or 60-80 hours, at a temperature of 20-80° C., preferably 40-60° C. or 50-60° C.

Preferably, the Bifidobacterium derived beta-galactosidase which has been modified by glycation has improved transgalactosylating activity as compared to an enzyme which has not been modified by glycation.

In preferred embodiment, the Bifidobacterium derived beta-galactosidase is selected from the group consisting of
(i) a beta-galactosidase
   a. having an amino acid sequence which is at least 50%, such as at least 60%, at least 70%, at least 80%, at least 90%, at least 95%, at least 98%, at least 99%, at least 99.5% or 100% identical to SEQ ID NO: 1,
   b. having a length of 850-1500 amino acids, preferably 1250-1500 amino acids, more preferably 1250-1350 or 1290-1350 amino acids, even more preferably 1300-1305 amino acids such as 1302 or 1304 amino acids, and
   c. having been modified by glycation, preferably by being contacted with 30-90 wt %, preferably 40-70 or 40-55 wt %, of a reducing sugar, preferably fructose, glucose, galactose or lactose, more preferably fructose, glucose or galactose, even more preferably glucose, at pH 4-7 for a time of 3-100 hours, preferably 40-100, 50-90 or 60-80 hours, at a temperature of 20-80° C., preferably 40-60° C. or 50-60° C.; and
(ii) a beta-galactosidase
   a. having an amino acid sequence which is at least 50%, such as at least 60%, at least 70%, at least 80%, at least 90%, at least 95%, at least 98%, at least 99%, at least 99.5% or 100% identical to SEQ ID NO: 2, and
   b. having a length of 850-1500 amino acids, preferably 850-1250 amino acids, more preferably 880-1215 amino acids, even more preferably 885-888 amino acids.

The Bifidobacterium derived beta-galactosidase may have been modified, such as chemically or enzymatically modified. Such modification may, e.g., have resulted in deamidation of certain amino acid residues. Such modification may give higher apparent MW in SDS-PAGE and/or change in isoelectric point. Chemical modification may have involved a thermal treatment. Such modification may have increased the ratio of transgalactosylation activity of the beta-galactosidase. Such modification may have made it easier to inactivate the beta-galactosidase, so that the modified beta-galactosidase may require a lower combination of high temperature and time for it to be inactivated compared to the same beta-galactosidase which has not been modified.

The beta-galactosidase having transgalactosylating activity may be extracellular. It may have had a signal sequence at its N-terminus, which was cleaved off during secretion.

The beta-galactosidase having transgalactosylating activity may be derived from any of the sources mentioned herein. The term “derived” means in this context that the enzyme may have been isolated from an organism where it is present natively, i.e. the identity of the amino acid sequence of the enzyme are identical to a native polypeptide. The term “derived” also means that the enzyme may have been produced recombinantly in a host organism, the recombinantly produced enzyme having either an identity identical to a native enzyme or having a modified amino acid sequence, e.g. having one or more amino acids which are deleted, inserted and/or substituted, i.e. a recombinantly produced enzyme which is a mutant and/or a fragment of a native amino acid sequence. Within the meaning of a native enzyme are included natural variants. Furthermore, the term “derived” includes enzymes produced synthetically by, e.g., peptide synthesis. The term “derived” also encompasses enzymes which have been modified e.g. by glycosylation, phosphorylation etc., whether in vivo or in vitro. With respect to recombinantly produced enzymes the term “derived from” refers to the identity of the enzyme and not the identity of the host organism in which it is produced recombinantly.

The beta-galactosidase having transgalactosylating activity may be obtained from a microorganism by use of any suitable technique. For instance, an enzyme preparation may be obtained by fermentation of a suitable microorganism and subsequent isolation of a beta-galactosidase preparation from the resulting fermented broth or microorganism by methods known in the art. The enzyme having transgalactosylating activity may also be obtained by use of recombinant DNA techniques. Such method normally comprises cultivation of a host cell transformed with a recombinant DNA vector comprising a DNA sequence encoding the lactase in question and the DNA sequence being operationally linked with an appropriate expression signal such that it is capable of expressing the enzyme in a culture medium under conditions permitting the expression of the enzyme and recovering the enzyme from the culture. The DNA sequence may also be incorporated into the genome of the host cell. The DNA sequence may be of genomic, cDNA or synthetic origin or any combinations of these, and may be isolated or synthesized in accordance with methods known in the art.

Preferably, the beta-galactosidase having transgalactosylating activity is purified.

In one aspect, the term “purified” as used herein refers to the beta-galactosidase having transgalactosylating activity being essentially free from insoluble components from the production organism. In other aspects. the term “purified” refers to the beta-galactosidase having transgalactosylating activity being essentially free from insoluble components from the native organism from which it is obtained. In one aspect, the beta-galactosidase having transgalactosylating activity is separated from some of the soluble components of the organism and culture medium from which it is derived. The beta-galactosidase having transgalactosylating activity may be purified (i.e. separated) by one or more of the unit operations filtration, precipitation, or chromatography.

Accordingly, the beta-galactosidase having transgalactosylating activity may be purified such that only minor amounts of other proteins, in particular other enzymes, are present. The term “purified” as used herein may refer to removal of other components, particularly other proteins and most particularly other enzymes present in the cell of origin of the beta-galactosidase. The betagalactosidase may be “substantially pure”, i.e. free from other components from the organism in which it is produced, i.e., e g, a host organism for recombinantly produced beta-galactosidase. In one aspect, the beta-galactosidase having transgalactosylating activity is at least 40% pure by weight of the total polypeptide material present in the preparation. In one aspect, the beta-galactosidase having transgalactosylating activity is at least 50%, 60%, 70%, 80% or 90% pure by weight of the total polypeptide material present in the preparation. As used herein. a “substantially pure enzyme” may denote an enzyme preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which the beta-galactosidase having transgalactosylating activity is natively or recombinantly associated.

It is, therefore, preferred that the substantially pure enzyme is at least 92% pure, preferably at least 94% pure, more preferably at least 95% pure, more preferably at least 96% pure, more preferably at least 97% pure, more preferably at least 98% pure, even more preferably at least 99% pure, most preferably at least 99.5% pure by weight of the total polypeptide material present in the preparation. The enzyme of the present invention is preferably in a substantially pure form (i.e., the preparation is essentially free of other polypeptide material with which it is natively or recombinantly associated). This can be accomplished, for example by preparing the polypeptide by well-known recombinant methods or by classical purification methods.

Preferably, the beta-galactosidase having transgalactosylating activity is isolated.

The term beta-galactosidases having transgalactosylating activity includes whatever auxiliary compounds may be necessary for the enzyme's catalytic activity, such as, e.g., an appropriate acceptor or cofactor, which may or may not be naturally present in the reaction system.

The beta-galactosidase may be in any form suited for the use in question, such as, e.g., in the form of a dry powder or granulate, a non-dusting granulate, a liquid, a stabilized liquid, or a protected enzyme.

In the methods of the invention, the Bifidobacterium derived beta-galactosidase is preferably added in an amount of 2,000-40,000, preferably 5,000-20,000, LAU(C) per liter milk-based substrate.

The activity in LAU(C) of a specific beta-galactosidase may be determined by direct measurement of glucose released from lactose. The skilled person will know how to determine such activity. Alternatively, the activity may be determined by using the activity assay described in the Methods and Examples of the present application. Here, the activity is obtained by comparing to a standard curve run with a beta-galactosidase of known activity, and the activity of the unknown sample calculated from this. A standard beta-galactosidase having a known LAU(C] activity can be supplied by Novozymes A/S.

### PREFERRED EMBODIMENTS

1. A method for producing a dairy product comprising galacto-oligosaccharides (GOS), said method comprising:
a. incubating a milk-based substrate with a Bifidobacterium derived beta-galactosidase having transgalactosylating activity to produce GOS, and
b. heat-treating the milk-based substrate comprising beta-galactosidase and GOS at a temperature of 72-78° C. for at least 12 minutes so that the residual activity of the beta-galactosidase is at most 0.1%.
   2. The method of embodiment 1, where in step a, the incubation is performed at 0-15° C., preferably at 4-10° C.
   3. The method of any of the preceding embodiments, where in step a, the incubation is performed for 12-30 hours, preferably for 16-25 hours.
   4. The method of embodiment 1, where in step a, the incubation is performed at 50-60° C., preferably at 56-60° C.
   5. The method of the preceding embodiment, where in step a, the incubation is performed for 30 minutes to 4 hours, preferably for 45 minutes to 2 hours.
   6. The method of any of the preceding embodiments, wherein the residual activity is the activity after step b in percent of the activity before step a.
   7. The method of any of the preceding embodiments, wherein after step a, the milk-based substrate comprises at least 10 wt % DP2+GOS, preferably at least 20 wt %, more preferably at least 30 wt % DP2+GOS, out of total carbohydrates.
   8. The method of any of the preceding embodiments, wherein after step a, the milk-based substrate comprises at least 10 wt % DP2 GOS, preferably at least 20 wt %, more preferably at least 30 wt % DP2 GOS, out of total carbohydrates.
   9. The method of any of the preceding embodiments, wherein after step a, the milk-based substrate comprises at least 1 wt % DP3+GOS, preferably at least 2 or at least 3 wt %, more preferably at least 4 or at least 5 wt % DP3+GOS, out of total carbohydrates.
   10. The method of any of the preceding embodiments, wherein after step a, the milk-based substrate comprises at least 1 wt % β-D-Galp-(1→3)-β-D-Galp-(1→4)-D-Glcp GOS, preferably at least 2 or at least 3 wt %, more preferably at least 4 or at least 5 wt % β-D-Galp-(1→3)-β-D-Galp-(1→4)-D-Glcp GOS, out of total carbohydrates.
   11. The method of any of the preceding embodiments, wherein after step a, the milk-based substrate comprises 10-70 wt % DP2+GOS, preferably 20-60 wt %, more preferably 30-50 wt % DP2+GOS, out of total carbohydrates.
   12. The method of any of the preceding embodiments, wherein after step a, the milk-based substrate comprises 10-70 wt % DP2 GOS, preferably 20-60 wt %, more preferably 25-40 wt % DP2 GOS, out of total carbohydrates.
   13. The method of any of the preceding embodiments, wherein after step a, the milk-based substrate comprises 1-50 wt % DP3+GOS, preferably 2-40 wt %, more preferably 5-30 wt % DP3+GOS, out of total carbohydrates.
   14. The method of any of the preceding embodiments, wherein after step a, the milk-based substrate comprises 1-30 wt % β-D-Galp-(1→3)-β-D-Galp-(1→4)-D-Glcp GOS, preferably 2-20 wt %, more preferably 3-15 wt % β-D-Galp-(1→3)-β-D-Galp-(1→4)-D-Glcp GOS, out of total carbohydrates.
   15. The method of any of the preceding embodiments, where in step b, the heat treatment is performed at a temperature of 73-78° C., preferably 73-77° C., more preferably 74-77° C., even more preferably 74-76° C., and most preferably 74.5-75.5° C.
   16. The method of any of the preceding embodiments, where in step b, the heat treatment is performed for at least 13 minutes, preferably at least 14 minutes, more preferably at least 15 minutes, even more preferably at least 30 minutes, most preferably at least 45 minutes.
   17. The method of any of the preceding embodiments, where in step b, the heat treatment is performed for 12 minutes to 6 hours, such as for 13 minutes, 14 minutes, 15 minutes, 30 minutes or 45 minutes to 6 hours, preferably for 12 minutes to 4 hours, such as for 13 minutes, 14 minutes, 15 minutes, 30 minutes or 45 minutes to 4 hours, more preferably for 12 minutes to 2 hours, such as for 13 minutes, 14 minutes, 15 minutes, 30 minutes or 45 minutes to 2 hours, even more preferably for 12 minutes to 1 hour, such as for 13 minutes, 14 minutes, 15 minutes, 30 minutes or 45 minutes to 1 hour, and most preferably for 30 minutes to 1 hour.
   18. The method of any of the preceding embodiments, where in step b, the heat treatment is performed for the time it takes to fill and empty the tank in which the heat treatment takes place.
   19. The method of any of the preceding embodiments, wherein after step b, preferably immediately after step b, the residual activity of the Bifidobacterium derived beta-galactosidase is at most 0.05%, preferably at most 0.01%.
   20. The method of any of the preceding embodiments, wherein after step b, the amount of β-D-Galp-(1→3)-β-D-Galp-(1→4)-D-Glcp GOS is reduced by at most 15%, preferably at most 10%, after 3 months storage of the dairy product at 20° C.
   21. A method for producing a dairy product comprising galacto-oligosaccharides (GOS), said method comprising:
a. adding to a milk-based substrate a Bifidobacterium derived beta-galactosidase having transgalactosylating activity, and
b. incubating at a temperature of 62-66° C., preferable 62.5-65.5° C., more preferably 63-65° C., for at least 2 hours, preferably at least 3 hours, more preferably at least 4 hours, to produce GOS and at the same time inactivate the beta-galactosidase so that after the incubation, the residual activity of the beta-galactosidase is at most 1%, preferably at most 0.5%.
   22. The method of the preceding embodiment, wherein the residual activity is the activity after step b in percent of the activity before step a.
   23. The method of any of the two preceding embodiments, wherein after step b, the milk-based substrate comprises at least 8 wt % DP2+GOS, preferably 8-70 wt %, more preferably 8-50 wt % DP2+GOS, out of total carbohydrates.
   24. The method of any of the three preceding embodiments, wherein after step b, the milk-based substrate comprises at least 2 wt % DP2 GOS, preferably 2-60 wt %, more preferably 2-40 wt % DP2 GOS, out of total carbohydrates.
   25. The method of any of the four preceding embodiments, wherein after step b, the milk-based substrate comprises at least 5 wt % DP3+GOS, preferably 5-50 wt %, more preferably 5-30 wt % DP3+GOS, out of total carbohydrates.
   26. The method of any of the five preceding embodiments, wherein after step b, the milk-based substrate comprises at least 1 wt % β-D-Galp-(1→3)-β-D-Galp-(1→4)-D-Glcp GOS, preferably 1-30 wt %, more preferably 3-15 wt % R-D-Galp-(1→3)-β-D-Galp-(1→4)-D-Glcp GOS, out of total carbohydrates.
   27. The method of any of the six preceding embodiments, wherein step b is performed for 2-6 hours, preferably 3-5 hours.
   28. The method of any of the preceding embodiments, wherein the dairy product is milk, e.g., skim milk, low fat milk, whole milk, UHT milk, ESL milk or HTST milk, or chocolate milk or yoghurt or whey.
   29. The method of any of the preceding embodiments, wherein the milk-based substrate comprises lactose, preferably 3-10 wt % lactose, more preferably 4-5.5 wt % lactose.
   30. The method of any of the preceding embodiments, wherein the milk-based substrate is milk, e.g., raw milk, skim milk, low fat milk or whole milk, reconstituted milk powder, e.g., reconstituted skim milk powder, or chocolate milk or whey.
   31. The method of any of the preceding embodiments, wherein the milk-based substrate is obtained from raw milk and has not been largely modified in composition by addition and/or withdrawal of constituents except for adjustment of fat content and/or water.
   32. The method of any of the preceding embodiments, wherein before step a, the milk-based substrate has been pasteurized.
   33. The method of any of the preceding embodiments, wherein the Bifidobacterium derived beta-galactosidase is derived from Bifidobacterium bifidum.
   34. The method of any of the preceding embodiments, wherein the Bifidobacterium derived beta-galactosidase has been C-terminally truncated compared to the wild-type enzyme from which is it derived.
   35. The method of any of the preceding embodiments, wherein the Bifidobacterium derived beta-galactosidase has a length of 850-1500 amino acids, preferably 850-1350 amino acids, more preferably 880-1350, 880-1320 or 885-1310 amino acids.
   36. The method of any of the preceding embodiments, wherein the Bifidobacterium derived beta-galactosidase has been modified, such as chemically or enzymatically modified.
   37. The method of the preceding embodiment, wherein the modification increases the ratio of transgalactosylation activity of the beta-galactosidase.
   38. The method of any of the two preceding embodiments, wherein the modification makes it easier to inactivate the beta-galactosidase, preferably wherein the modified beta-galactosidase can be inactivated at lower temperature compared to the same beta-galactosidase which has not been modified.
   39. The method of any of the preceding embodiments, wherein the Bifidobacterium derived beta-galactosidase has an amino acid sequence which is at least 50%, such as at least 60%, at least 70%, at least 80%, at least 90%, at least 95%, at least 98%, at least 99%, at least 99.5% or 100% identical to any of SEQ ID NOs: 1 or 2.
   40. The method of any of the preceding embodiments, wherein the Bifidobacterium derived beta-galactosidase has an amino acid sequence which is at least 50%, such as at least 60%, at least 70%, at least 80%, at least 90%, at least 95%, at least 98%, at least 99%, at least 99.5% or 100% identical to SEQ ID NO: 1.
   41. The method of the preceding embodiment, wherein the Bifidobacterium derived beta-galactosidase has a length of 850-1500 amino acids, preferably 1250-1500 amino acids, more preferably 1250-1350 or 1290-1350 amino acids, even more preferably 1300-1305 amino acids such as 1302 or 1304 amino acids.
   42. The method of any of the preceding embodiments, wherein the Bifidobacterium derived beta-galactosidase has been modified by glycation of at least one lysine and/or arginine residue.
   43. The method of any of the preceding embodiments, wherein the Bifidobacterium derived beta-galactosidase has been contacted with a reducing sugar, preferably fructose, glucose, galactose, or lactose, for a time and temperature sufficient to produce a polypeptide modified by glycation.
   44. The method of any of the preceding embodiments, wherein the Bifidobacterium derived beta-galactosidase has been modified by glycation by contacting with 30-90 wt %, preferably 40-70 or 40-55 wt %, of a reducing sugar, preferably fructose, glucose, galactose or lactose, more preferably fructose, glucose or galactose, even more preferably glucose, at pH 4-7 for a time of 3-100 hours, preferably 40-100, 50-90 or 60-80 hours, at a temperature of 20-80° C., preferably 40-60° C. or 50-60° C.
   45. The method of any of the three preceding embodiments, wherein the Bifidobacterium derived beta-galactosidase which has been modified by glycation has improved transgalactosylating activity as compared to an enzyme which has not been modified by glycation.
   46. The method of any of the preceding embodiments, wherein the Bifidobacterium derived beta-galactosidase has an amino acid sequence which is at least 50%, such as at least 60%, at least 70%, at least 80%, at least 90%, at least 95%, at least 98%, at least 99%, at least 99.5% or 100% identical to SEQ ID NO: 2.
   47. The method of any of the preceding embodiments, wherein the Bifidobacterium derived beta-galactosidase has a length of 850-1500 amino acids, preferably 850-1250 amino acids, more preferably 880-1215 amino acids, even more preferably 885-888 amino acids.
   48. The method of any of the preceding embodiments, wherein the Bifidobacterium derived beta-galactosidase has a ratio of transgalactosylation activity of at least 150%.
   49. The method of the preceding embodiment, wherein the ratio of transgalactosylation activity is determined according to Method 3 of WO2013182686.
   50. The method of any of the preceding embodiments, wherein the Bifidobacterium derived beta-galactosidase has a ratio of transgalactosylating to hydrolysing activity of at least 1, preferably at least 2, at least 3 or at least 4, when measured in a 60% solution of skim milk powder after 24 hours at 5° C. using an enzyme concentration of 350 LAU(C) per g lactose.
   51. The method of the preceding embodiment, wherein the ratio of transgalactosylating to hydrolysing activity is calculated as ([Glc]˜[Gal])/[Gal], where [Glc] is the relative concentration of glucose and [Gal] is the relative concentration of galactose after the skim milk powder solution and the beta-galactosidase has been incubated for 24 hours at 5° C.
   52. The method of the preceding embodiment, wherein the concentration of glucose and galactose, respectively, is measured using HPLC.
   53. The method of any of the preceding embodiments, wherein the Bifidobacterium derived beta-galactosidase belongs to the enzyme class 3.2.1.23.
   54. The method of any of the preceding embodiments, wherein the Bifidobacterium derived beta-galactosidase is isolated.
   55. The method of any of the preceding embodiments, wherein the Bifidobacterium derived beta-galactosidase is purified.
   56. The method of any of the preceding embodiments, wherein the Bifidobacterium derived beta-galactosidase is added in an amount of 2,000-40,000, preferably 5,000-20,000, LAU(C) per liter milk-based substrate.
   57. The method of any of the preceding embodiments, wherein after step b, an ESL, an HTST or a UHT treatment is performed.
   58. The method of any of the preceding embodiments, wherein after step b, the dairy product is stored at below 10° C., such as at 0-10° C.
   59. The method of any of the preceding embodiments, wherein after step b, a drying, preferably a spray-drying, is performed to obtain a dried dairy product such as a milk powder.
   60. The method of any of the preceding embodiments, wherein the dairy product is used in the production of a protein bar, a milk drink, an infant formula or a yoghurt.
   61. The method of any of the preceding embodiments, wherein after step b, the dairy product is fermented with a bacterial culture to provide a fermented dairy product comprising GOS.
   62. The method of the preceding embodiment, wherein the fermentation with the bacterial culture is performed at a temperature of 37-45° C.

### EXAMPLES

### Materials and Methods

### Beta-galactosidase Having Transgalactosylating Activity

Saphera® Fiber (Novozymes A/S) having the sequence shown as SEQ ID NO: 1 is a glycated form of Bifidobacterium bifidum derived beta-galactosidase having a declared activity of 3000 LAU(C)/g. Glycation increases the transgalactosylating ability of the enzyme and can be performed, e.g., as described in the examples of WO 2018/210821.

Nurica® is a commercial GOS-producing (i.e., transgalactosylating) Bifidobacterium bifidum derived beta-galactosidase product from IFF expectedly comprising BIF917 disclosed in, e.g., WO2013182686 and having the sequence shown as SEQ ID NO: 2.

Both of these beta-galactosidase products have a ratio of transgalactosylation activity as determined according to Method 3 of WO2013182686 of above 150%.

### Activity Assay (LAU(C))

### Principle:

Lactase hydrolyzes lactose to form α-D-glucose. The α-D-glucose is phosphorylated by ATP, in a reaction catalyzed by hexokinase. The glucose-6-phosphate formed is oxidized to 6-phosphogluconate by glucose-6-phosphate dehydrogenase. Concomitant with this reaction an equimolar amount of NAD+ is reduced to NADH with a resulting increase in absorbance at 340 nm.

### Reagents:

15% (w/v) Brij L23: Weigh out 508.0±0.4 g of Brij® L23 (Sigma B4184) into a beaker. Add approx. 300 mL ultrapure water and stir. Transfer the Brij® L23 quantitatively to a 1 L volumetric flask. Fill to the mark with ultrapure water. Stir until homogenous. Storability: 2 months in refrigerator.

Colour reagent: (Glucose reagent kit (GHK) (0.1 M Tris, 2.1 mM ATP, 2.1 mM NAD, 4 mM Mg2+, <0.1% NaN3, 4 mMMg2+, >7.5 kU/L hexokinase, >7.5 kU/L G-6-P-DH, pH 7.8)): Open a vial of Glucose (HK) Reagent A, Thermo Fisher Scientific (Art. no.: 981304 or 981779) and a vial of Glucose (HK) Reagent B, Thermo Fisher Scientific (Art. no.: 981304 or 981779). Pour 1 vial of reagent B into 1 vial of reagent A. Put on the cap. Mix well by slowly and gently turning up and down the vial 10-15 times. Use the whole mixture in reagent A vial, or pour needed amount into an appropriate container. Storability: 1 month in refrigerator.

Dissolution buffer/dilution buffer (0.01 M Citric acid monohydrate, 0.0225% (w/v) Brij® L23, 1 mM MgSO4, 7H2O, pH 4.5): Weigh out 21.0±0.1 g of Citric acid monohydrate (Cas. No. 5949-29-1) and transfer quantitative to a 10 L volumetric flask. Weigh out 2.46±0.01 g of MgSO4, 7H2O (Cas. no. 10034-99-8) and transfer quantitative to the volumetric flask. Add approximately 9 L of demineralized water and stir until completely dissolved. Add 15 mL of 15% (w/v) Brij L23 to the volumetric flask and stir. Add approximately 35 mL of 4 M NaOH (Cas. No. 1310-73-2) and stir. Adjust pH to 4.50±0.05 using e.g. 4 M NaOH or e.g. HCl as appropriate. Fill to the mark with demineralized water and stir. Storability: 13 days at room temperature.

Substrate (31.6% w/w lactose monohydrate, 0.01M citric monohydrate, 0.0225 (w/v) Brij L23, 1 mM MgSO4, 7H2O): Weigh out 7.9±0.2 g of Lactose monohydrate (Cas. No. 10039-26-6) directly into a beaker. Dissolve to a total volume of 25.0±0.1 g of dissolution buffer. Heat up and stir until fully dissolved with no boiling of the substrate. Storability: 6 hours at room temperature.

Standard: Enzyme standard with identified LAU(C)/g (available from Novozymes A/S, Denmark) is used as standard, diluted in dissolution buffer in the range from 0.197-0.7880 LAU(C)/mL.

### Procedure:

1. 50 μL of substrate is incubated for 540 seconds at 50° C. Blank (50 uL of dissolution buffer) is subtracted out.
2. 25 uL sample in dissolution buffer is added.
3. The reaction is incubated for 1800 seconds followed by addition of 160 uL colour reagent.
4. After 300 seconds, the absorbance is measured at 340 nm.

### Calculation of Enzyme Activity:

The enzyme activity of the diluted sample is read from the standard curve. Calculation of activity of a sample in LAU(C)/g is as stated in the formula:

$$Activity Unit / g = \frac{S \cdot V \cdot F}{W}$$

S=Reading from the standard curve in LAU(C)/mL
V=Volume of the measuring flask used in mL
F=Dilution factor for second dilution
W=Weight of sample in g

### Example 1

Heat Treatment of GOS-Enriched Milk with Enzyme at 70-90° C. for 5-15 Minutes

2.3% (w/v) Saphera Fiber corresponding to 69,000 LAU(C) per L milk was added to skimmed milk having a lactose content of 4.7%. This is a 10-fold higher enzyme dosage compared to the standard dosage recommended. The overdosing is done in order to accelerate the impact of residual activity and hereby in a convenient way shorten the laboratory storage time. When the enzyme is overdosed 10-fold, the residual activity will also be 10-fold higher. Thus, 14 days storage with 10× dose is comparable to 140 days storage with 1× dose.

The milk and enzyme was incubated for 30 min at 20° C. (room temperature) to make a GOS enriched milk. Then samples of 1.5 ml of this solution were incubated at 70° C., 75° C., 80° C. and 90° C. for 5 min, 8 min, 12 min and 15 min at each temperature. A control sample was also collected without heat treatment. This control sample and all heat-treated samples were placed in dry ice for quick freezing just after each incubation.

Note: Here, the residual activity is determined as the activity after the heat treatment in percent of the activity after the incubation (30 min at 20° C.) to produce GOS but before the heat treatment. But since the incubation at 20° C. for 30 min will not contribute to inactivation of the enzyme, the same residual activity would have been obtained had it been determined as the activity after the heat treatment in percent of the activity before the incubation (30 min at 20° C.) to produce GOS.

### Residual Activity Assay

Samples were centrifuged at 20,600 g in a precooled centrifuge for 45 min at 5° C. and dilution with 20 mM sodium succinic acid and 0.01% triton x-100, pH 6.5 of the supernatant was made so an absorbance reading below 1.5 at 405 nm could be measured. Twenty five μl of each sample was mixed with 175 μl of ONPG substrate (1.67 mg/ml ONPG (o-NitroPhenyl β-D-galatopyranoside, ˜5.5 mM), 0.05 M MES, 1 mM MgSO4, 150 mM KCl, 0.01% Triton X-100, pH 6.5) and incubated for 2.5 hr at 40° C. and stopped by adding 50 μl Na2CO3+5 mM Na4EDTA and measured at 405 nm. Residual activity in % was calculated using the formulae=((Abs405^{heat treated sample}−Abs405^{blank})*dilution factor)/((Abs405^{untreated sample}−Abs405^{blank})*dilution factor))*100%.

Table 1 shows the residual activity as percent of activity without heat treatment (average of 2 experiments). Limit of Quantitation, LoQ, is 0.01% residual activity.

TABLE 1

Avg.

| | 70° C. | 75° C. | 80° C. | 90° C. |
|---|---|---|---|---|
| Minutes | % | % | % | % |
| 5 | 44.95 | 12.44 | 0.10 | 0.022 |
| 8 | 2.02 | 0.032 | 0.015 | 0.050 |
| 12 | 0.065 | <LoQ | 0.021 | 0.053 |
| 15 | 0.018 | <LoQ | 0.027 | 0.048 |

The residual activity declined as expected with increasing temperatures and increasing incubation time. Very low residual activity is needed to achieve a stable GOS content in a final dairy product which is to be stored for prolonged time at ambient temperature, and for such dairy products, an enzyme inactivation to at least below 0.1% residual activity is typically needed depending on the shelf life of the dairy product. Surprisingly, inactivation at 90° C. and 80° C. results in minimum residual activity when inactivating for approximately 5 minutes and 8 minutes, respectively, and then higher residual activity is seen when inactivating for a longer time at these temperatures. Whereas inactivation at 70° C. and 75° C. results in a continuous decrease in residual activity when increasing the inactivation time from 5 minutes to 15 minutes. Especially, inactivation at 75° C. results in a very low residual activity when using an inactivation time of at least 12 minutes. Low residual activity of the enzyme is crucial for GOS enriched dairy products to have a long shelf life and ensure a good safety margin as process variations in production can happen. Furthermore, lower inactivation temperatures are generally preferred to avoid off-flavors of the final dairy product.

### Example 2

Heat treatment of GOS-enriched milk with enzyme at 75° C. for 5-60 minutes

2.3% (w/v) Saphera Fiber corresponding to 69,000 LAU(C) per L milk was added to skimmed milk having a lactose content of 4.7%. This is a 10-fold higher enzyme dosage compared to the standard dosage recommended. The overdosing is done in order to accelerate the impact of residual activity and hereby in a convenient way shorten the laboratory storage time. When the enzyme is overdosed 10-fold, the residual activity will also be 10-fold higher. Thus, 14 days storage with 10× dose is comparable to 140 days storage with 1× dose.

The milk and enzyme was incubated for 30 min at 20° C. (room temperature) to make a GOS enriched milk. Then samples of 1.5 ml of this solution were incubated at 75° C. for 5 min, 15 min, 30 min and 60 min. A control sample was also collected without heat treatment. Furthermore, samples which had been heat inactivated for 60 min were incubated for 3 days or 14 days at room temperature (20° C.). At the end of each incubation 10 ul acetic acid was added followed by heating to 90° C. for 5 min and centrifugation at max. rpm for 5 min at room temp. 1000 ul supernatant was transferred to an Eppendorf tube and GOS determination was made using HPLC.

### Residual Activity Assay

Samples were centrifuged at 20,600 g in a precooled centrifuge for 45 min at 5° C. and dilution with 20 mM sodium succinic acid and 0.01% triton x-100, pH 6.5 of the supernatant was made so an absorbance reading below 1.5 at 405 nm could be measured. Twenty five μl of each sample was mixed with 175 μl of ONPG substrate (1.67 mg/ml ONPG (o-NitroPhenyl β-D-galatopyranoside, ˜5.5 mM), 0.05 M MES, 1 mM MgSO4, 150 mM KCl, 0.01% Triton X-100, pH 6.5) and incubated for 24 hr at 40° C. and stopped by adding 50 μl Na2CO3+5 mM Na4EDTA and measured at 405 nm. Residual activity in % was calculated using the formulae=((Abs405^{heat treated sample}−Abs405^{blank})*dilution factor)/((Abs405^{untreated sample}−Abs405^{blank})*dilution factor))*100%.

GOS determination, ECD-HPLC, PA1 column

50 μl sample was mixed with 500 ul Milli Q water and 10 μl Carrez I solution in a 5 ml Eppendorf tube. Then 10 ul Carrez II solution was added and mixed. Finally, 4.43 ml milli Q water was added and centrifuged at 20,600 g for 5 min at room temp. 1 ml of supernatant was transferred to a new tube and 4 ml water added and then filtered through a 0.20 μm filter into an HPLC vial.

TABLE 2
Amount of lactose (Lac), disaccharide GOS (all disaccharide
except lactose) (DP2G), and trisaccaride β-D-Galp-(1→3)-
β-D-Galp-(1→4)-D-Glcp (the dominant trisaccharide GOS) (Gal_{—}
b1-3_Lac) in % of total carbohydrate after enzyme inactivation
at 75° C. for the indicated time followed by storage
for 3 and 14 days at 20° C. (average of 2 experiments)

| | 3 days storage at 20° C. | | | 14 days storage at 20° C. | | |
|---|---|---|---|---|---|---|
| Inacti- | | | Gal_b1- | | | Gal_b1- |
| vation time | Lac | DP2G | 3_Lac | Lac | DP2G | 3_Lac |
| min | % | % | % | % | % | % |
| 5 | 0.1 | 1.6 | 0.0 | 0.1 | 1.8 | 0.0 |
| 15 | 7.3 | 20.8 | 3.5 | 7.1 | 25.3 | 3.4 |
| 30 | 7.3 | 21.2 | 3.4 | 7.2 | 25.9 | 3.7 |
| 60 | 7.4 | 21.3 | 3.7 | 9.4 | 25.3 | 4.3 |

TABLE 3
Residual activity (average of 2 experiments). Limit
of Quantitation, LoQ, is 0.01% residual activity

| 0 min | 5 min | 15 min | 30 min | 60 min |
|---|---|---|---|---|
| % | % | % | % | % |
| 100 | 27.6 | <LoQ | <LoQ | <LoQ |

In Table 2, the lactose level is the primary indicator of residual enzyme activity. After inactivation at 75° C. for 15, 30 and 60 minutes, only a small decline in lactose is seen from 3 days to 14 days of storage. From this it can reasonably be concluded that also for the first 3 days, there will be only little decline if any. The higher lactose seen for 60 minutes inactivation after 14 days of storage is an experimental outlier, since the lactose level cannot increase.

After inactivation at 75° C. for 15, 30 and 60 minutes, no decline in GOS is seen from 3 days to 14 days of storage. The somewhat higher DP2G levels seen after 14 days of incubation compared to 3 days is expected to result from to day to day variation regarding the efficiency of separating the various DP2G peaks in HPLC. The higher Gal_b1-3_Lac seen for 60 minutes inactivation after 14 days of storage is expected to be an experimental outlier.

The enzyme dosage used is approximately 10× overdosed according to recommended application, i.e. suggesting that this experiment would match a 30 days (3 days×10) or 140 days (14 days×10) storage, respectively, at room temperature.

The GOS made is not stable after only 5 minutes' incubation at 75° C. showing that the enzyme is not sufficiently inactivated. Under these conditions, all Gal_b1-3_Lac and almost all DP2G has been degraded already after 3 days (corresponding to 30 days) storage.

### Example 3

### GOS Production at 56-60° C. Using Saphera Fiber

Incubation of Enzyme with Milk at Different Temperatures

0.23% (w/v) Saphera Fiber (corresponding to 6,900 LAU(C) per L milk) was added to skimmed milk. Samples of 1.5 ml were transferred to Eppendorf tubes and incubated at 56° C., 58° and 60° C. for 1 h, 2 h, 4 h and 22 h at 1000 rpm in an Eppendorf thermomixer. After incubation the samples were frozen below −18° C. until analysis using HPAEC-PAD and HPSEC-RI.

### HPAEC-PAD

High-Performance Anion-Exchange Chromatography with Pulsed Amperometric Detection (HPAEC-PAD) using a PA1 column for quantitative determination of Gal, Glc, Lac and DP2 GOS's as well as selected DP3+GOS's. The method is used to determine the ratio of lactose to DP2-GOS which is used for the quantification of the amount of lactose in DP2 determined by HPSEC-RI.

50 μl sample was mixed with 500 ul Milli Q water and 10 μl Carrez I solution in a 5 ml Eppendorf tube. Then 10 ul Carrez II solution was added and mixed. Finally, 4.43 ml milli Q water was added and centrifuged at 20,600 g for 5 min at room temp. 1 ml of supernatant was transferred to a new tube and 4 ml water added and then filtered through a 0.20 μm filter into an HPLC vial.

### HPSEC-RI

Inactivation of samples was achieved by diluting the 1 ml sample with 9 ml 0.04 M NaOH and incubate for 5 min at room temperature. Carbohydrate analysis (quantification of galacto-oligosaccharides (DP3+), DP2 (lactose+DP2-GOS), glucose and galactose) was performed by HPLC by applying the samples on a Dionex HPLC system ICS-5000 RI (Aminex-HPX-87H Ion Exclusion Column) with 5 mM H2SO4 as isocratic mobile phase.

TABLE 4
(average of 2 experiments)

| | | |
|---|---|---|
| 56° C. | 58° C. | 60° C. |

| | Lac | DP3+ | Lac | DP3+ | Lac | DP3+ |
|---|---|---|---|---|---|---|
| hours | % | % | % | % | % | % |
| 1 | 30.7 | 26.2 | 34.4 | 26.2 | 43.2 | 24.4 |
| 2 | 15.9 | 25.8 | 23.6 | 28.5 | 36.8 | 26.8 |
| 4 | 5.5 | 15.5 | 15.6 | 28.1 | 32.9 | 27.7 |
| 22 | 0.4 | 3.5 | 11.3 | 26.1 | 31.4 | 28.2 |

### Incubation at 56-60° C. can be Used to Generate GOS.

### Example 4

### “One Step GOS Production and Inactivation” Application at 61-65° C.

The purpose of this experiment was to investigate if incubation of milk with Saphera Fiber or Nurica to produce GOS at 61-65° C. for sufficiently long time would at the same time inactivate the enzyme thus enabling a “one step GOS production and inactivation” application.

Incubation of Enzyme with Milk at Different Temperatures

0.23% (w/v) Saphera Fiber (corresponding to 6,900 LAU(C) per L milk) or 0.23% (w/w) Nurica was added to skimmed milk. Aliquots of 1.5 ml were transferred to Eppendorf tubes and incubated at 61° C., 63° and 65° C. for 1 h, 2 h and 4 h at 1000 rpm in an Eppendorf thermomixer. A control sample was also collected without incubation. Storage tests were also made by storing 4 h heat treated samples for 1 week, 2 weeks and 4 weeks at room temperature (23° C.). After incubation, the samples were frozen below −18° C. until analysis using HPAEC-PAD as described in Example 3 and for residual activity.

### Residual Activity Assay

Samples were centrifuged at 20,600 g in a precooled centrifuge for 45 min at 5° C. and dilution with 20 mM sodium succinic acid and 0.01% triton x-100, pH 6.5 of the supernatant was made so an absorbance reading below 1.5 at 405 nm could be measured. Twenty five μl of each sample was mixed with 175 μl of ONPG substrate (1.67 mg/ml ONPG (o-NitroPhenyl β-D-galatopyranoside, ˜5.5 mM), 0.05 M MES, 1 mM MgSO4, 150 mM KCl, 0.01% Triton X-100, pH 6.5) and incubated for 20 hr at 40° C. and stopped by adding 50 μl Na2CO3+5 mM Na4EDTA and measured at 405 nm. Residual activity in % was calculated using the formulae=((Abs405 heat treated sample−Abs405^{blank})*dilution factor)/((Abs405^{untreated sample}−Abs405^{blank})*dilution factor))*100%.

TABLE 5
(average of 2 experiments)

| | Saphera Fiber, 61° C. | | | Saphera Fiber, 63° C. | | | Saphera Fiber, 65° C. | | |
|---|---|---|---|---|---|---|---|---|---|
| Incuba- | | | Gal_b1- | | | Gal_b1- | | | Gal_b1- |
| tion time | Lac | DP2G | 3_Lac | Lac | DP2G | 3_Lac | Lac | DP2G | 3_Lac |
| hour | % | % | % | % | % | % | % | % | % |
| 1 | 43.5 | 10.5 | 9.7 | 54.6 | 7.8 | 8.1 | 65.0 | 4.7 | 6.6 |
| 2 | 40.2 | 11.1 | 9.6 | 53.8 | 8.1 | 9.1 | 65.0 | 4.8 | 7.1 |
| 4 | 38.1 | 12.2 | 10.5 | 53.4 | 8.3 | 9.2 | 64.6 | 5.0 | 6.9 |

| Storage | | | Gal_b1- | | | Gal_b1- | | | Gal_b1- |
|---|---|---|---|---|---|---|---|---|---|
| time | Lac | DP2G | 3_Lac | Lac | DP2G | 3_Lac | Lac | DP2G | 3_Lac |
| weeks | % | % | % | % | % | % | % | % | % |
| 1 | 27.5 | 12.1 | 11.2 | 52.3 | 9.9 | 9.6 | 63.4 | 7.3 | 7.6 |
| 2 | 19.2 | 12.7 | 11.3 | 50.7 | 10.0 | 9.6 | 61.7 | 7.6 | 7.7 |
| 4 | 8.0 | 13.7 | 8.5 | 47.2 | 10.6 | 9.8 | 58.8 | 8.2 | 7.9 |

TABLE 6
(average of 2 experiments)

| | Nurica, 61° C. | | | Nurica, 63° C. | | | Nurica, 65° C. | | |
|---|---|---|---|---|---|---|---|---|---|
| Incuba- | | | Gal_b1- | | | Gal_b1- | | | Gal_b1- |
| tion time | Lac | DP2G | 3_Lac | Lac | DP2G | 3_Lac | Lac | DP2G | 3_Lac |
| hour | % | % | % | % | % | % | % | % | % |
| 1 | 57.1 | 6.9 | 9.2 | 68.1 | 3.5 | 7.2 | 73.6 | 2.2 | 5.8 |
| 2 | 54.1 | 7.9 | 10.1 | 67.5 | 3.8 | 7.5 | 73.4 | 2.2 | 5.9 |
| 4 | 52.0 | 8.6 | 10.4 | 67.3 | 4.0 | 7.7 | 73.1 | 2.4 | 6.2 |

| Storage | | | Gal_b1- | | | Gal_b1- | | | Gal_b1- |
|---|---|---|---|---|---|---|---|---|---|
| time | Lac | DP2G | 3_Lac | Lac | DP2G | 3_Lac | Lac | DP2G | 3_Lac |
| weeks | % | % | % | % | % | % | % | % | % |
| 1 | 42.7 | 12.1 | 11.0 | 65.5 | 6.4 | 8.3 | 72.1 | 4.9 | 6.4 |
| 2 | 36.0 | 14.2 | 11.5 | 63.8 | 7.0 | 8.5 | 71.5 | 5.1 | 6.4 |
| 4 | 26.1 | 16.6 | 12.7 | 60.9 | 8.0 | 9.0 | 70.7 | 5.4 | 6.7 |

TABLE 7
Residual activities (average of 2 experiments). Limit
of Quantitation, LoQ, is 0.01% residual activity

| | Saphera Fiber | | | Nurica | | |
|---|---|---|---|---|---|---|
| | 61° C. | 63° C. | 65° C. | 61° C. | 63° C. | 65° C. |
| hours | % | % | % | % | % | % |
| 1 | 19.75 | 1.39 | 0.05 | 22.67 | 4.14 | 0.63 |
| 2 | 4.53 | 0.26 | <LoQ | 7.61 | 0.86 | 0.27 |
| 4 | 0.50 | <LoQ | <LoQ | 2.07 | 0.20 | <LoQ |

In Tables 5 and 6, the lactose level is the primary indicator of residual enzyme activity. The levels of DP2G and Gal_b1-3_Lac merely shows that as long as there is a small amount of residual enzyme activity, the GOS level increases until the level of lactose is below 10-20%

Table 5 shows that for Saphera Fiber, when incubating at 63° C., the lactose level is almost stable from 2 hours to 4 hours of incubation going from 53.8% to 53.4%, i.e. less than 1% degradation. When incubating at 65° C., the lactose level is almost stable from 1 to 2 hours and also from 2 to 4 hours. From 2 hours to 4 hours of incubation, it drops from 65.0% to 64.6%, i.e. less than 1% degradation. Table 7 shows that Saphera Fiber is inactivated to below 1% residual activity after 4 hours at 61° C., after 2 hours at 63° C. and after 1 hour at 65° C. Table 5 further shows that after 4 hours of incubation at 61° C., the lactose level has dropped from 38.1% to 8.0% (79% degradation) after 4 weeks of storage, whereas after 4 hours of incubation at 63° C. or 65° C., only 12% and 9%, respectively, of the lactose has been degraded after 4 weeks of storage (63° C.: 53.4% to 47.2%; 65° C.: 64.6% to 58.8%).

Table 6 shows that also for Nurica, when incubating at 63° C., the lactose level is almost stable from 2 hours to 4 hours of incubation going from 67.5% to 67.3%, i.e. less than 1% degradation. When incubating at 65° C., the lactose level is almost stable from 1 to 2 hours and also from 2 to 4 hours. From 2 hours to 4 hours of incubation, it drops from 73.4% to 73.1%, i.e. less than 1% degradation. Table 7 shows that Nurica is inactivated to below 1% residual activity after 2 hours at 63° C. and after 1 hour at 65° C. Table 6 further shows that after 4 hours of incubation at 61° C., the lactose level has dropped from 52.0% to 26.1% (50% degradation) after 4 weeks of storage, whereas after 4 hours of incubation at 63° C. or 65° C., only 10% and 3%, respectively, of the lactose has been degraded after 4 weeks of storage (63° C.: 67.3% to 60.9%; 65° C.: 73.1% to 70.7%).

From these data it is concluded that below 1% residual enzyme activity after a “one step GOS production and inactivation” process step is sufficient to ensure an acceptably low lactose degradation and no or limited GOS degradation for some weeks of storage at 23° C. This is useful in applications where the dairy product is not to be stored for prolonged time at ambient temperature or where a later process step is included which will further inactivate the enzyme, e.g., a UHT treatment or a drop in pH such as in yoghurt production. A “one step GOS production and inactivation” at at least 63° C. for at least 2 hours is therefore useful in such applications.

### Example 5

### GOS Production at 56° C. Followed by Inactivation at 75° C.

The purpose of this experiment was to make GOS in milk at elevated temperature to shorten incubation time (to save tank capacity), here for 1 hour, followed by an inactivation step at 75° C. for 30 min or 60 min. Thus, the whole process including inactivation is below 2 hours compared to standard incubation for 24 hours at 5° C. (which further needs an inactivation step).

0.23% (w/v) Saphera Fiber (corresponding to 6,900 LAU(C) per L milk) was added to skimmed milk with 0.025% sodium azide. Samples of 1.2 ml were transferred to Eppendorf tubes and incubated at 56° C. for 1 h at 1000 rpm in an Eppendorf thermomixer. Then the samples were incubated at 30 min or 60 min at 75° C. and then stored at 5° C. or 23° C. for one week. A control sample was also collected without incubation at 56° C. and heat treatment. Samples were collected throughout the experiment and frozen below −18° C. until analysis using HPAEC-PAD and HPSEC-RI and determination of residual activity.

### HPAEC-PAD

High-Performance Anion-Exchange Chromatography with Pulsed Amperometric Detection (HPAEC-PAD) using a PA1 column for quantitative determination of Gal, Glc, Lac and DP2 GOS's as well as selected DP3+GOS's. The method is used to determine the ratio of lactose to DP2-GOS which is used for the quantification of the amount of lactose in DP2 determined by HPSEC-RI. 50 μl sample was mixed with 500 ul Milli Q water and 10 μl Carrez I solution in a 5 ml Eppendorf tube. Then 10 ul Carrez II solution was added and mixed. Finally, 4.43 ml milli Q water was added and centrifuged at 20,600 g for 5 min at room temp. 1 ml of supernatant was transferred to a new tube and 4 ml water added and then filtered through a 0.20 μm filter into an HPLC vial.

### HPSEC-RI

Inactivation of samples was achieved by diluting the 1 ml sample with 9 ml 0.04 M NaOH and incubate for 5 min at room temperature. Carbohydrate analysis (quantification of galacto-oligosaccharides (DP3+), DP2 (lactose+DP2-GOS), glucose and galactose) was performed by HPLC by applying the samples on a Dionex HPLC system ICS-5000 RI (Aminex-HPX-87H Ion Exclusion Column) with 5 mM H2SO4 as isocratic mobile phase.

Residual activity was determined as in Example 4.

Table 8 Relative carbohydrate content after incubation with Saphera Fiber (SF) for 60 min at 56° C. followed by an inactivation treatment for 30 min at 75° C. Samples were analyzed just after 75° C. treatment or after one week stored at 5° C. or 23° C.

| | Inactivation, SF | | |
|---|---|---|---|
| | Lac | DP2G | DP3+ |
| 75° C. for 30 min | % | % | % |
| No storage | 35.3 | 13.3 | 24.4 |
| 1 week, 5° C. | 32.5 | 14.9 | 24.8 |
| 1 week, 23° C. | 22.0 | 20.1 | 27.2 |

Table 9 Relative carbohydrate content after incubation with Saphera Fiber (SF) for 60 min at 56° C. followed by an inactivation treatment for 60 min at 75° C. Samples were analyzed just after 75° C. treatment or after one week stored at 5° C. or 23° C.

| | Inactivation, SF | | |
|---|---|---|---|
| | Lac | DP2G | DP3+ |
| 75° C. for 60 min | % | % | % |
| No storage | 35.1 | 13.4 | 24.4 |
| 1 week, 5° C. | 34.6 | 13.4 | 24.6 |
| 1 week, 23° C. | 28.8 | 16.0 | 26.1 |

Table 10 Relative carbohydrate content after incubation with Nurica (N) for 60 min at 56° C. followed by an inactivation treatment for 30 min at 75° C. Samples were analyzed just after 75° C. treatment or after one week stored at 5° C. or 23° C.

| | Inactivation, N | | |
|---|---|---|---|
| | Lac | DP2G | DP3+ |
| 75° C. for 30 min | % | % | % |
| No storage | 37.4 | 13.3 | 28.0 |
| 1 week, 5° C. | 36.2 | 14.1 | 28.1 |
| 1 week, 23° C. | 33.2 | 12.3 | 30.5 |

Table 11 Relative carbohydrate content after incubation with Nurica (N) for 60 min at 56° C. followed by an inactivation treatment for 60 min at 75° C. Samples were analyzed just after 75° C. treatment or after one week stored at 5° C. or 23° C.

| | Inactivation, N | | |
|---|---|---|---|
| | Lac | DP2G | DP3+ |
| 75° C. for 60 min | % | % | % |
| No storage | 37.3 | 13.3 | 28.1 |
| 1 week, 5° C. | 37.0 | 12.9 | 28.3 |
| 1 week, 23° C. | 33.1 | 14.7 | 29.3 |

Table 12 Residual activity after enzymatic treatment at 56° C. and after 75° C. inactivation step. LoQ is 0.01% residual activity.

| | | 56° C./1 h + | 56° C./1 h + |
|---|---|---|---|
| | 56° C./1 h | 75° C./30 min | 75° C./60 min |
| | % | % | % |
| Saphera Fiber | 69 | <LoQ | <LoQ |
| Nurica | 61 | <LoQ | <LoQ |

The enzyme is inactivated at 75° C. for 30 or 60 minutes to a very low residual level.

In Tables 8-11, the lactose level is the primary indicator of residual enzyme activity. Some lactose degradation is seen upon storage for a week.

The level of GOS fiber (DP3+) does not decline during storage, which is the most important parameter for commercial application of the process.

The slight general increase in the levels of DP2G and DP3+ merely shows that as long as there is a small amount of residual enzyme activity, the GOS level increases until the level of lactose is below 10-20%.

GOS production at 56° C. for 1 hour followed by inactivation at 75° C. for 30 or 60 minutes is shown here to be a viable commercial process where the enzyme is inactivated to very low residual activity and DP3+GOS does not decline after one week's storage. However, for dairy products having an extended storage time, i.e. several weeks/months, inactivation for 60 min at 75° C. and having low storage temperature can be chosen to give higher certainty for GOS fiber stability.

1. A method for producing a dairy product comprising galacto-oligosaccharides (GOS), said method comprising:
a. incubating a milk-based substrate with a Bifidobacterium derived beta-galactosidase having transgalactosylating activity to produce GOS, and
b. heat-treating the milk-based substrate comprising beta-galactosidase and GOS at a temperature of 72-78° C. for at least 12 minutes so that the residual activity of the beta-galactosidase is at most 0.1%.

2. The method of claim 1, where in step a, the incubation is performed at 0-15° C., for 12-30 hours.

3. The method of claim 1, where in step a, the incubation is performed at 50-60° C., for 30 minutes to 4 hours.

4. The method of claim 1, where in step b, the heat treatment is performed at a temperature of 73-78° C.

5. The method of claim 1, where in step b, the heat treatment is performed for at least 13 minutes.

6. The method of claim 1, wherein after step b, the residual activity of the Bifidobacterium derived beta-galactosidase is at most 0.05%

7. A method for producing a dairy product comprising galacto-oligosaccharides (GOS), said method comprising:
a. adding to a milk-based substrate a Bifidobacterium derived beta-galactosidase having transgalactosylating activity, and
b. incubating at a temperature of 62-66° C., for at least 2 hours, to produce GOS and at the same time inactivate the beta-galactosidase so that after the incubation, the residual activity of the beta-galactosidase is at most 1%.

8. The method of claim 7, wherein step b is performed for 2-6 hours.

9. The method of claim 1, wherein the residual activity is the activity after step b, in percent of the activity before step a.

10. The method of claim 1, wherein the Bifidobacterium derived beta-galactosidase is derived from Bifidobacterium bifidum.

11. The method of claim 1, wherein the Bifidobacterium derived beta-galactosidase has been C-terminally truncated compared to the wild-type enzyme from which is it derived to have a length of 850-1500 amino acids.

12. The method of claim 1, wherein the Bifidobacterium derived beta-galactosidase has an amino acid sequence which is at least 50% identical to SEQ ID NOs: 1 or 2.

13. The method of claim 1, wherein the Bifidobacterium derived beta-galactosidase is selected from the group consisting of:
(i) a beta-galactosidase
a. having an amino acid sequence which is at least 50% identical to SEQ ID NO: 1,
b. having a length of 850-1500 amino acids, and
c. having been modified by glycation, of a reducing sugar at pH 4-7 for a time of 3-100 hours, at a temperature of 20-80° C.; and
(ii) a beta-galactosidase
a. having an amino acid sequence which is at least 50% identical to SEQ ID NO: 2, and
b. having a length of 850-1500 amino acids.

14. The method of claim 1, wherein the Bifidobacterium derived beta-galactosidase has a ratio of transgalactosylation activity of at least 150%.

15. The method of claim 1, wherein after step b, the dairy product is stored at below 10° C.
